(19) United States
(12) Patent Application Publication
(10) Pub. No.: US 2026/0144234 A1
(43) Pub. Date: May 28, 2026

(54) Stretchable Waist Velcro For Diapers

(71) Applicant: U-PLAY CORPORATION, Wuhu, Anhui (CN)

(72) Inventors: Sheng Cheng, Wuhu, Anhui (CN); Jing Jin, Wuhu, Anhui (CN); Wenguang Yang, Wuhu, Anhui (CN)

(21) Appl. No.: 18/989,964

(22) Filed: Dec. 20, 2024

(30) Foreign Application Priority Data
Nov. 22, 2024 (CN) 2024116827342

(51) Int. Cl. A01K 23/00; A61F 13/56

(52) U.S. Cl. CPC A01K 23/00; A61F 13/56; A61F 2013/5694

(57) ABSTRACT

A stretchable waist Velcro for diapers, comprising a diaper body, wherein the diaper body is provided with elastic belts and Velcro for adaptively wearing the diaper body on pets of different sizes; the diaper body is provided with stretchable belts for keeping an elastic structure in close contact with a pet's belly, and the elastic belt is provided with a transmission mechanism for controlling the retraction and expansion of the stretchable belt. When the pet's stomach is full and bulging, the belly will become larger and bulge outward, so the elastic belt will be stretched, but using the elastic belt to adapt to the pet's enlarged belly will cause the pet's belly to be strangled. Through the movement of multiple stretching blocks and patches, and the V-shaped setting of the stretching belt, the stretching block and the patch will unfold the V-shaped stretching belt.

### TECHNICAL FIELD

The invention related to the technical field of diapers, and in particular to a stretchable waist Velcro for diapers.

### BACKGROUND ART

Pet diapers are a type of diaper specially designed for pets. They are mostly used to control the urination and defecation of dogs, which can prevent dogs from urinating and defecating randomly, and can also facilitate the handling of female dogs' menstrual period.

During the use of existing pet diapers, a layer of hair of varying thickness will grow on the surface of the pet, and the hair will reduce the roughness of the surface. Therefore, when the diaper is fixed with an elastic belt, the restraint is generally high and relatively tight. However, in actual use, the pet's abdomen changes (for example, when the pet finishes eating, the abdomen will bulge to a certain extent. At this time, the elastic belt, which is already tight, will be pulled tighter, giving the pet a bad experience). Therefore, the elastic restraint of the elastic belt needs to be adjusted, and the adjustment can be made automatically. Secondly, during the use of the diaper, when a certain amount of urine is collected inside, the weight of the diaper will increase. At this time, when the pet is moving, the diaper may fall down (on the pet, it will move back and loosen), which results in a poor experience for the pet when wearing the diaper, and the diaper is prone to fall off as the weight increases.

### SUMMARY OF THE INVENTION

The technical solution of the invention is aimed at the technical problem that the existing technical solutions are too single, and provides a solution that is significantly different from the existing technical solutions. It mainly provides a stretchable waist Velcro for diapers to solve the above problems in the background art. The technical problem raised is that pets have a poor experience wearing diapers when their stomachs bulge after eating, and the diapers tend to fall off as they become heavier.

The technical solution adopted by the invention to solve the above technical problems is:

A stretchable waist Velcro for diapers, comprising a diaper body, wherein the diaper body is provided with elastic belts and Velcro for adaptively wearing the diaper body on pets of different sizes; the diaper body is provided with stretchable belts for keeping an elastic structure in close contact with a pet's belly, and the elastic belt is provided with a transmission mechanism for controlling the retraction and expansion of the stretchable belt.

Preferably, both sides of the diaper body are provided with ring bodies, and the ring body is provided with a stretchable belt.

Preferably, the stretchable belt is provided with an elastic belt, the elastic belt is provided with a Velcro, and the Velcro on both sides are attached to each other.

Preferably, the transmission mechanism comprises a pulling belt, which is arranged on the elastic belt, and an end of the elastic belt away from the pulling belt is provided with a limiting ring.

Preferably, the stretchable belt is composed of a plurality of V-shaped structures; the stretchable belt is provided with a plurality of stretching blocks, and patches and connecting rods are provided between the plurality of stretching blocks; an end of the pulling belt away from the elastic belt passes through the limiting ring and is connected to the stretching blocks.

Preferably, the pulling belt is provided with a tensioning ring, which is arranged on both sides of the limiting ring; the tensioning ring is provided with a connecting belt, and an end of the connecting belt away from the tensioning ring is connected to the pet's clothes.

Compared with the prior art, the beneficial effects of the invention are as follows: an elastic belt is provided on the diaper body, and a Velcro is provided at one end of the elastic belt. When the diaper body is worn on a dog, the elastic belts on both sides are stretched so that the Velcro on the elastic belts on both sides fit together to achieve wearing. The setting of the elastic belt allows the diaper body to adapt to pets of different sizes, so that the situation of buying a diaper body too large or too small to be unsuitable for the pet will not occur.

When the pet's stomach is full and bulging, the belly will become larger and bulge outward, so the elastic belt will be stretched, but using the elastic belt to adapt to the pet's enlarged belly will cause the pet's belly to be strangled (because the elastic belt stretches to adapt to the pet's belly through elastic deformation, and the longer it is stretched, the greater the force it will generate to strangle the dog's belly). However, at this time, the elastic belt is stretched and the stretching block is pulled by the pulling belt. Through the movement of multiple stretching blocks and patches, and the V-shaped setting of the stretching belt, the stretching block and the patch will unfold the V-shaped stretching belt. As the stretching belt unfolds to replace the stretching of the elastic belt, the elastic belt will shrink a part until the elastic belt and the stretching belt are in a balanced state, which can reduce the elastic deformation of a part of the elastic belt. In this way, as the pet's belly becomes larger, it does not rely entirely on the elastic belt to stretch, reducing the force of the elastic belt stretching on the pet's belly, the pet's belly will not be strangled very seriously, reducing the discomfort of the pet's belly when it bulges, and as the pet's belly becomes smaller, the elastic belt and the stretching belt will continue to shrink to keep the elastic belt part in contact with the pet's belly, which also prevents the diaper body from loosening after the pet's belly bulges and then becomes smaller.

A connecting belt is provided on the pet's clothes. When the diaper body becomes heavier and the pet runs, it moves backward. At this time, the tensioning ring will pull the pulling belt forward, and cooperate with the limiting ring to lock the pulling belt. At this time, since the pulling belt is restricted, the elastic belt and the stretchable belt will not be easily deformed. In this way, the part of the elastic belt that fits the pet's belly cannot stretch and expand, resulting in the elastic belt being unable to adapt to the enlargement of the pet's belly (because the elastic belt part of the diaper body is worn on the front part of the pet's belly, and the pet's belly is bigger the further back it is, the backward movement of the diaper body requires the elastic belt that fits the belly to be expanded before it can move backward). At this time, the elastic belt cannot be easily expanded, and the connecting belt has the function of pulling the diaper body. In this way, after the diaper body becomes heavier, it cannot move backward due to restriction, and it will not easily move and fall off.

The invention will be described in detail hereinafter with reference to the drawings and specific embodiments.

### BRIEF DESCRIPTION OF THE DRAWINGS

FIG. 1 is a schematic diagram of the front three-dimensional structure according to the invention;

FIG. 2 is a schematic diagram of a top view of the three-dimensional structure according to the invention;

FIG. 3 is a schematic diagram of a side view of the three-dimensional structure according to the invention;

FIG. 4 is a schematic diagram of an outer structure of the stretchable belt and the elastic belt according to the invention;

FIG. 5 is a schematic diagram of an inner structure of the stretchable belt and the elastic belt according to the invention;

FIG. 6 is a schematic diagram of the three-dimensional structure of the stretching structure according to the invention;

FIG. 7 is a schematic diagram of the enlarged structure of part A in FIG. 2;

FIG. 8 is a schematic diagram of the enlarged structure of part B in FIG. 5;

In the figures:

1 refers to the diaper body; 2 refers to the ring body; 3 refers to the elastic belt; 4 refers to the Velcro; 5 refers to the stretchable belt; 6 refers to the stretching block; 7 refers to the patch; 8 refers to the connecting rod; 9 refers to the pulling belt; 10 refers to the limiting ring; 11 refers to the connecting belt; 12 refers to the tensioning ring.

### SPECIFIC EMBODIMENT OF THE INVENTION

To facilitate understanding of the invention, the invention will be described more comprehensively hereinafter with reference to the drawings. Several embodiments of the invention are given in the drawings, but the invention can be implemented in different forms and is not limited thereto. On the contrary, these embodiments are provided to make the content disclosed in the invention more thorough and comprehensive.

It should be noted that when an element is referred to as being “fixed on” another element, it may be directly on the other element or there may be a central element. When an element is considered to be “connected to” another element, it may be directly connected to the other element or there may be a central element at the same time. The terms “vertical”, “horizontal”, “left”, “right” and similar expressions used in this article are for illustrative purposes only.

Unless otherwise defined, all technical and scientific terms used herein have the same meaning as commonly connected by technicians in the technical field to which the invention belongs. The terminology used in the specification of the invention is for the purpose of describing specific embodiments and is not intended to limit the invention. The term “and/or” used herein includes any and all combinations of one or more related listed items.

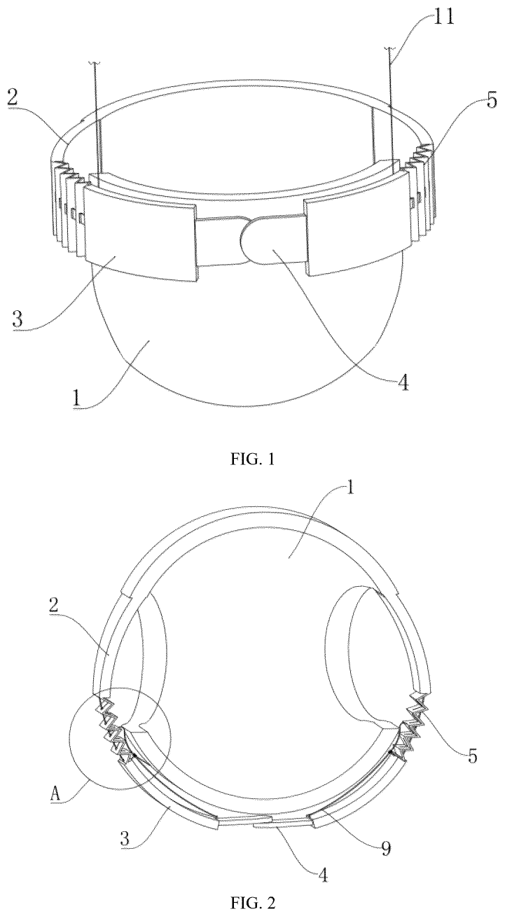

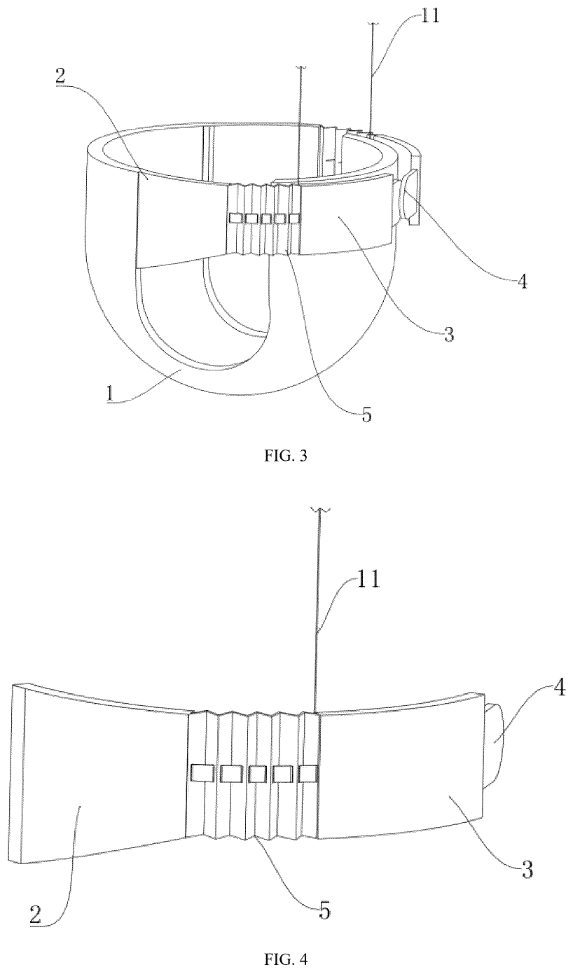

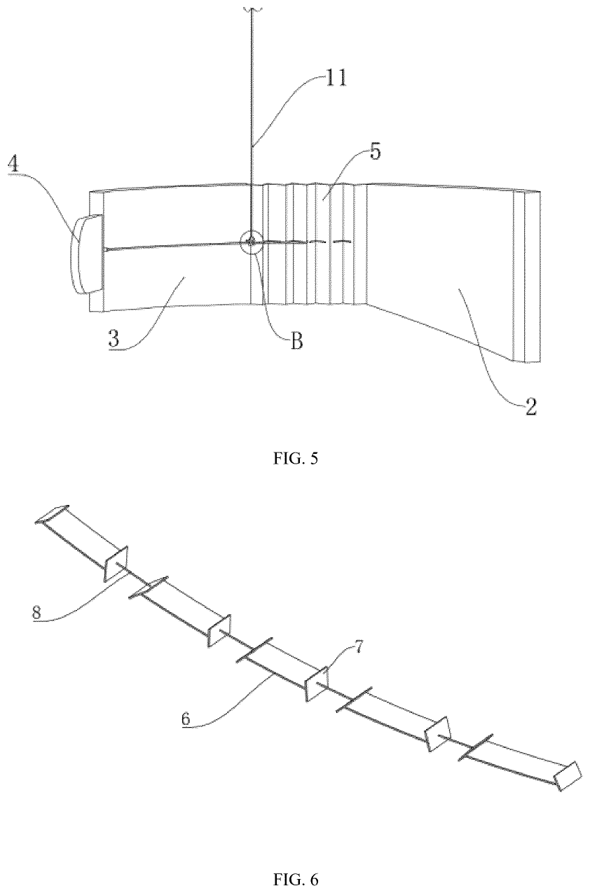

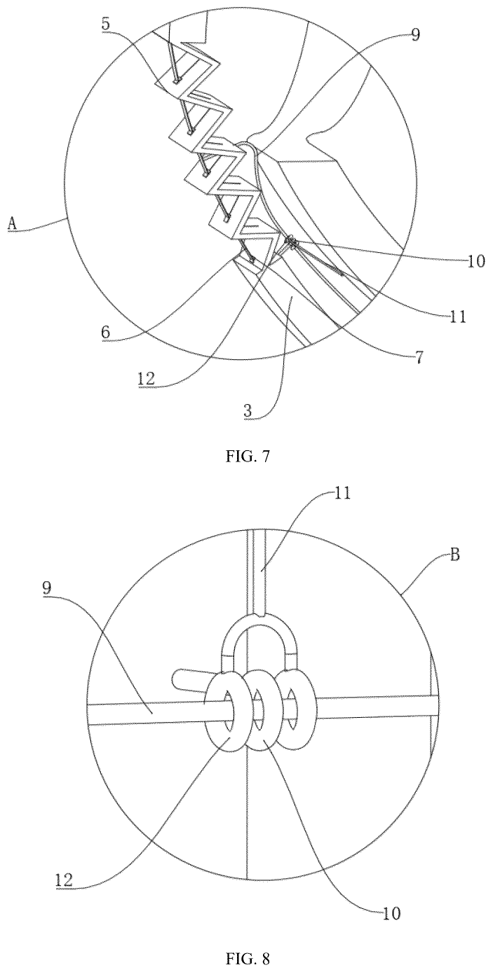

With reference to FIGS. 1-8, a stretchable waist Velcro for diapers, comprising a diaper body 1, wherein the diaper body 1 is provided with elastic belts 3 and Velcro 4 for adaptively wearing the diaper body 1 on pets of different sizes; the diaper body 1 is provided with stretchable belts 5 for keeping an elastic structure in close contact with a pet's belly, and the elastic belt 3 is provided with a transmission mechanism for controlling the retraction and expansion of the stretchable belt 5.

The specific operation process of the invention is as follows: wear the diaper body 1 on the pet's buttocks, then stretch the Velcro 4 outwards until adhered to each other and fit together, and fit the elastic belt 3 to the front part of the pet's belly and fix it. At this time, the diaper body 1 is worn on the pet. When the pet's belly becomes bigger after eating, the pet's bulging belly will push the elastic belt 3 outward. At this time, the elastic belts 3 on both sides will be stretched first, and the stretching of the elastic belt 3 will drive the stretchable belt 5 to stretch through the transmission mechanism. As the stretching of the stretchable belt 5 replaces the elastic stretching of the elastic belt 3, the elastic belt 3 begins to shrink partly until the elastic belt 3 and the stretchable belt 5 are in a balanced state. As the food in the pet's belly is digested, the pet's belly becomes smaller, and the elastic belt 3 and the stretchable belt 5 will also shrink as the pet's belly becomes smaller, so as to keep the elastic belt 3 always fit on the dog's belly.

With reference to FIGS. 1-3, both sides of the diaper body 1 are provided with ring bodies 2, and the ring body 2 is provided with a stretchable belt 5; the stretchable belt 5 is provided with an elastic belt 3, the elastic belt 3 is provided with a Velcro 4, and the Velcro 4 on both sides are attached to each other.

When wearing the diaper body 1, the ring bodies 2 on both sides are wrapped around the pet's belly, and then the Velcro 4 on both sides are pulled outward. The Velcro 4 will pull the elastic belt 3 to stretch until the Velcro 4 on both sides fit together, and then the diaper body 1 is worn on the pet through the Velcro 4 sticking to each other. Due to the setting of the elastic belt 3, the elastic belt 3 can fit on the pet's belly, so that pets of different sizes can be worn and fixed.

With reference to FIGS. 2-7, the transmission mechanism comprises a pulling belt 9, which is arranged on the elastic belt 3, and an end of the elastic belt 3 away from the pulling belt 9 is provided with a limiting ring 10; the stretchable belt 5 is composed of a plurality of V-shaped structures; the stretchable belt 5 is provided with a plurality of stretching blocks 6, and patches 7 and connecting rods 8 are provided between the plurality of stretching blocks 6; an end of the pulling belt 9 away from the elastic belt 3 passes through the limiting ring 10 and is connected to the stretching blocks 6.

When the elastic belt 3 is stretched, the elastic belt 3 stretches and pulls the pulling belt 9, which is made of a material with a certain hardness such as rubber or plastic. The pulling belt 9 pulls one of the stretching blocks 6, and one of the stretching blocks 6 is cooperated with the patch 7 and the connecting rod 8 to drive the entire group of stretching blocks 6, the patch 7 and the connecting rod 8 to move. The patch 7 moves along the V-shaped inner wall of the stretchable belt 5. The stretchable belt 5 is composed of composite cardboard. As the patch 7 continues to move, the stretchable belt 5 is pushed to continue to expand, which makes the stretchable belt 5 longer. As the stretchable belt 5 becomes longer, the elastic belt 3 contracts through its own elasticity to keep fitting with the pet's belly. When the balance point is reached, the elastic belt 3 and the stretchable belt 5 stop stretching, and the elastic belt 3 is in a state of fitting with the pet's belly. However, since a part of the stretching of the elastic belt 3 is replaced by the stretchable belt 5, the elastic belt 3 is not so tight on the pet's belly.

With reference to FIG. 9, the pulling belt 9 is provided with a tensioning ring 12, which is arranged on both sides of the limiting ring 10; the tensioning ring 12 is provided with a connecting belt 11, and an end of the connecting belt 11 away from the tensioning ring 12 is connected to the pet's clothes.

The connecting belt 11 is provided on the pet's clothes. When the diaper body 1 is changed and worn on the pet, the tensioning ring 12 at one end of the connecting belt 11 is provided on the pulling belt 9, and the two tensioning rings 12 are provided on both sides of the limiting ring 10. When the diaper body 1 moves backward, the tensioning rings 12 are cooperated with the limiting ring 10 to pull the pulling belt 9 into a V shape, so that the pulling belt 9 can no longer move, thereby limiting the elastic belt 3 and the telescopic belt 5, and the pulling belt 9 itself will also have a pulling effect on the backward moving diaper body 1 to prevent the diaper body 1 from moving backward and falling off after becoming heavy. The connecting belt 11 is not a disposable item and will not be replaced with the diaper body 1, so it can be used many times for a long time.

The above is an exemplary description of the invention in combination with the drawings. It is obvious that the specific implementation of the invention is not limited to the above method. As long as such non-substantial improvements are made by adopting the method concept and technical solution of the invention, or the concept and technical solution of the invention are directly applied to other occasions without improvement, they are all within the protection scope of the invention.

1. A stretchable waist Velcro for diapers, comprising a diaper body, wherein the diaper body is provided with elastic belts and Velcro for adaptively wearing the diaper body on pets of different sizes; the diaper body is provided with stretchable belts for keeping an elastic structure in close contact with a pet's belly, and the elastic belt is provided with a transmission mechanism for controlling the retraction and expansion of the stretchable belt.

2. The stretchable waist Velcro for diapers of claim 1, wherein both sides of the diaper body are provided with ring bodies, and the ring body is provided with a stretchable belt.

3. The stretchable waist Velcro for diapers of claim 1, wherein the stretchable belt is provided with an elastic belt, the elastic belt is provided with a Velcro, and the Velcro on both sides are attached to each other.

4. The stretchable waist Velcro for diapers of claim 1, wherein the transmission mechanism comprises a pulling belt, which is arranged on the elastic belt, and an end of the elastic belt away from the pulling belt is provided with a limiting ring.

5. The stretchable waist Velcro for diapers of claim 4, wherein the stretchable belt is composed of a plurality of V-shaped structures; the stretchable belt is provided with a plurality of stretching blocks, and patches and connecting rods are provided between the plurality of stretching blocks; an end of the pulling belt away from the elastic belt passes through the limiting ring and is connected to the stretching blocks.

6. The stretchable waist Velcro for diapers of claim 4, wherein the pulling belt is provided with a tensioning ring, which is arranged on both sides of the limiting ring; the tensioning ring is provided with a connecting belt, and an end of the connecting belt away from the tensioning ring is connected to the pet's clothes.
